SEQUENCE LISTING (10 sequences)

SEQ ID NO: 1 - protein, 10 aa, Artificial Sequence
  Synthetic construct
GFTFSDSWIH

SEQ ID NO: 2 - protein, 18 aa, Artificial Sequence
  Synthetic construct
AWISPYGGSTYYADSVKG

SEQ ID NO: 3 - protein, 9 aa, Artificial Sequence
  Synthetic construct
RHWPGGFDY

SEQ ID NO: 4 - protein, 11 aa, Artificial Sequence
  Synthetic construct
RASQDVSTAVA

SEQ ID NO: 5 - protein, 7 aa, Artificial Sequence
  Synthetic construct
SASFLYS

SEQ ID NO: 6 - protein, 9 aa, Artificial Sequence
  Synthetic construct
QQYLYHPAT

SEQ ID NO: 7 - protein, 118 aa, Artificial Sequence [sequence omitted]

SEQ ID NO: 8 - protein, 108 aa, Artificial Sequence
  Synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 9 - protein, 447 aa, Artificial Sequence
  Synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, Artificial Sequence
  Synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2019/0309071 A1
(43) Pub. Date: Oct. 10, 2019

(54) METHODS OF TREATING CANCER USING ANTI-PD-L1 ANTIBODIES AND ANTIANDROGENS

(71) Applicant: Genentech, Inc., South San Francisco, CA (US)

(72) Inventors: Sanjeev MARIATHASAN, Millbrae, CA (US); Christina SCHIFF, San Francisco, CA (US); Sujata NARAYANAN, Fremont, CA (US)

(21) Appl. No.: 16/437,402

(22) Filed: Jun. 11, 2019

Related U.S. Application Data
(63) Continuation of application No. PCT/US2017/065841, filed on Dec. 12, 2017.
(60) Provisional application No. 62/433,158, filed on Dec. 12, 2016.

(51) Int. Cl. C07K 16/28; C07K 14/705; A61P 35/02

(52) U.S. Cl. CPC C07K 16/2827; C07K 14/70532; C07K 2317/56; C07K 2317/524; A61P 35/02

(57) ABSTRACT

The present invention relates to the treatment of cancers, such as a prostate cancer (e.g., castration-resistant prostate cancer (CRPC)). More specifically, the invention concerns the treatment of human patients having a prostate cancer (e.g., CRPC, e.g., metastatic CRPC) with a combination therapy including an PD-1 axis binding antagonist and an antiandrogen.

### SEQUENCE LISTING

The instant application contains a Sequence Listing which has been submitted electronically in ASCII format and is hereby incorporated by reference in its entirety. Said ASCII copy, created on Jun. 5, 2019, is named 50474-153002 Sequence Listing_06.05.19_ST25 and is 9,565 bytes in size.

### FIELD OF THE INVENTION

The present invention relates to the treatment of cancers, such as prostate cancer (e.g., castration-resistant prostate cancer (CRPC)). More specifically, the invention concerns the treatment of human patients having a prostate cancer, such as metastatic CRPC (mCRPC) or locally confined, inoperable CRPC, by administering a combination of a PD-1 axis binding antagonist (e.g., an anti-PD-L1 antibody, e.g., atezolizumab) and an antiandrogen (e.g., enzalutamide).

### BACKGROUND

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. The National Cancer Institute has estimated that greater than half a million Americans will die of cancer in 2016, accounting for nearly one out of every four deaths in the country. As the elderly population has grown, the incidence of cancer has concurrently risen, as the probability of developing cancer is more than two-fold higher after the age of seventy. Cancer care thus represents a significant and ever-increasing societal burden.

With approximately 1.1 million newly diagnosed cases and more than 300,000 deaths each year worldwide, prostate cancer is the most commonly diagnosed cancer in men and the second leading cause of death in men in the Western world. The incidence rates are highest in developed regions, including North America and Australia.

Whereas most men with localized prostate cancer are cured with treatment, men with recurrent or newly diagnosed metastatic prostate cancer suffer significant morbidity and mortality. For patients who have recurrent prostate cancer following localized treatment and for those patients identified with de novo metastatic prostate cancer, the primary treatment is androgen deprivation therapy (ADT); however, up to one-third of prostate cancer patients will progress despite reduction in testosterone levels to castrate levels (<50 ng/dL) through surgical or medical castration. The majority of these men with castration-resistant prostate cancer (CRPC), and in particular metastatic CRPC (mCRPC), will experience deterioration in quality of life, disability, and ultimately die of their disease. The median life expectancy in patients diagnosed with mCRPC is less than three years, and less than one year in patients who have failed two prior lines of therapy.

Given the limitations of current treatments available for patients with prostate cancer (e.g., mCRPC), and particularly for prostate cancer (e.g., mCRPC) patients who have previously failed treatment with an androgen synthesis inhibitor and have failed, are ineligible for, or refused a taxane regimen, there remains an unmet need in the field for improved and tolerable treatment options for prostate cancer (e.g., mCRPC).

### SUMMARY OF THE INVENTION

The present invention relates to methods of treating a subject having cancer (e.g., prostate cancer, e.g., castration-resistant prostate cancer (CRPC), e.g., metastatic CRPC (mCRPC) or locally confined, inoperable CRPC) by administering a combination of a PD-1 axis binding antagonist (e.g., an anti-PD-L1 antibody, e.g., atezolizumab) and an antiandrogen (e.g., enzalutamide).

In one aspect, the invention features a method of treating a subject having a prostate cancer (e.g., CRPC) comprising administering to the subject an effective amount of an anti-PD-L1 antibody and an antiandrogen in one or more dosing cycles.

In some embodiments of the above aspect, the antiandrogen is an androgen receptor (AR) antagonist. In some embodiments, the AR antagonist is a non-steroidal AR antagonist. In some embodiments, the non-steroidal AR antagonist is enzalutamide. In some embodiments, the method comprises administering enzalutamide at a dose of between about 80 mg to about 240 mg. In some embodiments, the method comprises administering enzalutamide at a dose of about 160 mg. In some embodiments, the method comprises administering enzalutamide at a dose of about 160 mg on each day of the one or more dosing cycles.

In some embodiments of the above aspect, the anti-PD-L1 antibody inhibits the binding of PD-L1 to PD-1, the binding of PD-L1 to B7-1, or the binding of PD-L1 to both PD-1 and B7-1. In some embodiments, the anti-PD-L1 antibody is selected from the group consisting of atezolizumab (MPDL3280A), YW243.55.S70, MSB0010718C, MDX-1105, and MED14736. In some embodiments, the anti-PD-L1 antibody comprises the following hypervariable regions (HVRs): (a) an HVR-H1 sequence of GFTFSDSWIH (SEQ ID NO: 1), (b) an HVR-H2 sequence of AWISPYGGSTYYADSVKG (SEQ ID NO: 2), (c) an HVR-H3 sequence of RHWPGGFDY (SEQ ID NO: 3), (d) an HVR-L1 sequence of RASQDVSTAVA (SEQ ID NO: 4), (e) an HVR-L2 sequence of SASFLYS (SEQ ID NO: 5), and (f) an HVR-L3 sequence of QQYLYHPAT (SEQ ID NO: 6). In some embodiments, the anti-PD-L1 antibody comprises: (a) a heavy chain variable (VH) domain comprising an amino acid sequence having at least 95% sequence identity to the amino acid sequence of SEQ ID NO: 7, (b) a light chain variable (VL) domain comprising an amino acid sequence having at least 95% sequence identity to the amino acid sequence of SEQ ID NO: 8, or (c) a VH domain as in (a) and a VL domain as in (b). In some embodiments, the anti-PD-L1 antibody comprises: (a) a VH domain comprising the amino acid sequence of SEQ ID NO: 7, (b) a VL domain comprising the amino acid sequence of SEQ ID NO: 8, or (c) a VH domain as in (a) and a VL domain as in (b). In some embodiments, the anti-PD-L1 antibody comprises: (a) a VH domain comprising the amino acid sequence of SEQ ID NO: 7 and (b) a VL domain comprising the amino acid sequence of SEQ ID NO: 8.

In some embodiments of the above aspect, the anti-PD-L1 antibody is atezolizumab. In some embodiments, the method comprises administering atezolizumab at a dose of between about 600 mg to about 1800 mg. In some embodiments, the method comprises administering atezolizumab at a dose of between about 800 mg to about 1200 mg. In some embodiments, the method comprises administering atezolizumab at a dose of about 1200 mg. In some embodiments, the method comprises administering atezolizumab at a dose of about 5 mg/kg to about 20 mg/kg. In some embodiments, the method comprises administering atezolizumab at a dose of about 10 mg/kg to about 15 mg/kg. In some embodiments, the method comprises administering atezolizumab at a dose of about 15 mg/kg. In some embodiments, the method comprises administering atezolizumab at a fixed dose (e.g., a fixed dose of about 1200 mg or about 15 mg/kg).

In some embodiments of the above aspect, the method comprises administering the anti-PD-L1 antibody on about Day 1 of each of the one or more dosing cycles. In some embodiments, the length of each of the one or more dosing cycles is 18-24 days. In some embodiments, the length of each of the one or more dosing cycles is 21 days.

In some embodiments of the above aspect, the method comprises administering the antiandrogen before the anti-PD-L1 antibody, simultaneous with the anti-PD-L1 antibody, or after the anti-PD-L1 antibody. In some embodiments, the method comprises administering the anti-PD-L1 antibody intravenously, intramuscularly, subcutaneously, topically, orally, transdermally, intraperitoneally, intraorbitally, by implantation, by inhalation, intrathecally, intraventricularly, or intranasally. In some embodiments, the method comprises administering the anti-PD-L1 antibody intravenously. In some embodiments, the method comprises administering the antiandrogen orally, intravenously, intramuscularly, subcutaneously, topically, transdermally, intraperitoneally, intraorbitally, by implantation, by inhalation, intrathecally, intraventricularly, or intranasally. In some embodiments, the method comprises administering the antiandrogen orally.

In some embodiments of the above aspect, the method further comprises determining the expression level of a biomarker in a sample from the subject. In some embodiments, the biomarker is a T-effector-associated gene, an activated stroma-associated gene, or a myeloid-derived suppressor cell-associated gene. In some embodiments, the T-effector-associated gene is CD8A, perforin (PRF1), granzyme A (GZMA), granzyme B (GZMB), interferon-γ (IFN-γ), CXCL9, or CXCL10. In some embodiments, the activated stroma-associated gene is transforming growth factor-β (TGF-β), fibroblast-activated protein (FAP), podplanin (PDPN), a collagen gene, or biglycan (BGN). In some embodiments, the myeloid-derived suppressor cell-associated gene is CD68, CD163, FOXP3, or androgen-regulated gene 1. In some embodiments, the biomarker is PD-L1, CD8, or androgen receptor (AR) gene. In some embodiments, the biomarker is PD-L1. In some embodiments, a change in the expression level of the biomarker relative to a reference level is predictive of the subject's likelihood to respond to the treatment.

In some embodiments of the above aspect, the prostate cancer is a CRPC. In some embodiments, the CRPC is a metastatic CRPC (mCRPC). In some embodiments, the CRPC is a locally confined and inoperable CRPC.

In some embodiments of the above aspect, the subject failed to respond to a previous treatment comprising an androgen synthesis inhibitor. In some embodiments, the subject failed to respond to a previous treatment comprising an androgen synthesis inhibitor and a taxane regimen. In some embodiments, the subject failed to respond to a previous treatment comprising an androgen synthesis inhibitor and is ineligible for, or refuses treatment with, a taxane regimen. In some embodiments, the taxane regimen is for treatment of a hormone-sensitive prostate cancer or a castration-resistant prostate cancer. In some embodiments, the previous treatment comprising the androgen synthesis inhibitor was at least 28 days. In some embodiments, the androgen synthesis inhibitor is abiraterone, orteronel, galeterone, ketoconazole, or seviteronel.

In a second aspect, the invention features a kit comprising an anti-PD-L1 antibody and a package insert comprising instructions for administration of the anti-PD-L1 antibody in combination with an antiandrogen for treating a subject having a prostate cancer (e.g., a CRPC, e.g., mCRPC).

In a third aspect, the invention features a kit comprising a first medicament comprising an anti-PD-L1 antibody, a second medicament comprising an antiandrogen, and a package insert comprising instructions for administration of the first medicament and the second medicament for treating a subject having a prostate cancer (e.g., a CRPC, e.g., mCRPC).

In a fourth aspect, the invention features a kit comprising an antiandrogen and a package insert comprising instructions for administration of the antiandrogen in combination with an anti-PD-L1 antibody for treating a subject having a prostate cancer (e.g., a CRPC, e.g., mCRPC).

In some embodiments of the second, third, or fourth aspect, the antiandrogen is an AR antagonist. In some embodiments, the AR antagonist is a non-steroidal AR antagonist. In some embodiments, the non-steroidal AR antagonist is enzalutamide. In some embodiments, enzalutamide is formulated for administration at a dose of between about 80 mg to about 240 mg. In some embodiments, enzalutamide is formulated for administration at a dose of about 160 mg. In some embodiments, enzalutamide is formulated for administration at a dose of about 160 mg on each day of the one or more dosing cycles.

In some embodiments of the second, third, or fourth aspect, the anti-PD-L1 antibody inhibits the binding of PD-L1 to PD-1, the binding of PD-L1 to B7-1, or the binding of PD-L1 to both PD-1 and B7-1. In some embodiments, the anti-PD-L1 antibody is selected from the group consisting of atezolizumab (MPDL3280A), YW243.55.S70, MSB0010718C, MDX-1105, and MED14736. In some embodiments, the anti-PD-L1 antibody comprises the following HVRs: (a) an HVR-H1 sequence of GFTFSDSWIH (SEQ ID NO: 1), (b) an HVR-H2 sequence of AWISPYGGSTYYADSVKG (SEQ ID NO: 2), (c) an HVR-H3 sequence of RHWPGGFDY (SEQ ID NO: 3), (d) an HVR-L1 sequence of RASQDVSTAVA (SEQ ID NO: 4), (e) an HVR-L2 sequence of SASFLYS (SEQ ID NO: 5), and (f) an HVR-L3 sequence of QQYLYHPAT (SEQ ID NO: 6). In some embodiments, the anti-PD-L1 antibody comprises: (a) a heavy chain variable (VH) domain comprising an amino acid sequence having at least 95% sequence identity to the amino acid sequence of SEQ ID NO: 7, (b) a light chain variable (VL) domain comprising an amino acid sequence having at least 95% sequence identity to the amino acid sequence of SEQ ID NO: 8, or (c) a VH domain as in (a) and a VL domain as in (b). In some embodiments, the anti-PD-L1 antibody comprises: (a) a VH domain comprising the amino acid sequence of SEQ ID NO: 7, (b) a VL domain comprising the amino acid sequence of SEQ ID NO: 8, or (c) a VH domain as in (a) and a VL domain as in (b). In some embodiments, the anti-PD-L1 antibody comprises: (a) a VH domain comprising the amino acid sequence of SEQ ID NO: 7 and (b) a VL domain comprising the amino acid sequence of SEQ ID NO: 8.

In some embodiments of the second, third, or fourth aspect, the anti-PD-L1 antibody is atezolizumab. In some embodiments, atezolizumab is formulated for administration at a dose of between about 600 mg to about 1800 mg. In some embodiments, atezolizumab is formulated for administration at a dose of between 800 mg to about 1200 mg. In some embodiments, atezolizumab is formulated for administration at a dose of about 1200 mg. In some embodiments, atezolizumab is formulated for administration at a dose of about 5 mg/kg to about 20 mg/kg. In some embodiments, atezolizumab is formulated for administration at a dose of about 10 mg/kg to about 15 mg/kg. In some embodiments, atezolizumab is formulated for administration at a dose of about 15 mg/kg. In some embodiments, atezolizumab is formulated for administration at a fixed dose (e.g., a fixed dose of about 1200 mg or about 15 mg/kg).

In some embodiments of the second, third, or fourth aspect, the prostate cancer is a CRPC. In some embodiments, the CRPC is a metastatic CRPC. In some embodiments, the CRPC is a locally confined and inoperable CRPC. In some embodiments, the subject failed to respond to a previous treatment comprising an androgen synthesis inhibitor. In some embodiments, the subject failed to respond to a previous treatment comprising an androgen synthesis inhibitor and a taxane regimen. In some embodiments, the subject failed to respond to a previous treatment comprising an androgen synthesis inhibitor and is ineligible for, or refuses treatment with, a taxane regimen. In some embodiments, the taxane regimen is for treatment of a hormone-sensitive prostate cancer or a CRPC. In some embodiments, the previous treatment comprising the androgen synthesis inhibitor was at least 28 days. In some embodiments, the androgen synthesis inhibitor is abiraterone, orteronel, galeterone, ketoconazole, or seviteronel.

### DETAILED DESCRIPTION OF THE INVENTION

### I. Definitions

The term “about” as used herein refers to the usual error range for the respective value readily known to the skilled person in this technical field. Reference to “about” a value or parameter herein includes (and describes) embodiments that are directed to that value or parameter per se.

As used herein, “administering” is meant a method of giving a dosage of a compound (e.g., an anti-PD-L1 antibody and/or an antiandrogen) or a composition (e.g., a pharmaceutical composition, e.g., a pharmaceutical composition including an anti-PD-L1 antibody and/or an antiandrogen) to a subject. The compounds and/or compositions utilized in the methods described herein can be administered, for example, intravenously (e.g., by intravenous infusion), subcutaneously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The “amount” or “level” of a biomarker associated with an increased clinical benefit to a subject is a detectable level in a biological sample. These can be measured by methods known to one skilled in the art and also disclosed herein. The expression level or amount of biomarker assessed can be used to determine the response to the treatment.

The term “antiandrogen” refers to an agent that is capable of preventing or inhibiting the biologic effects of androgens (e.g., testosterone or dihydrotestosterone (DHT)) on normally responsive tissues in the body. Antiandrogens include agents that directly bind to and/or block the androgen receptor (AR) or one or more of its activities (e.g., AR antagonists), agents that directly inhibit the enzymatic biosynthesis of androgens (e.g., androgen synthesis inhibitors), and agents that suppress the gonadotropin-releasing hormone (GnRH)-induced release of gonadotropins (antigonadotropins). Antiandrogens may be steroidal or non-steroidal compounds. Steroidal antiandrogens include, but are not limited to, 17α-hydroxyprogesterone derivatives (e.g., chlormadinone acetate, cyproterone acetate, or megestrol acetate), 19-nortestosterone derivatives (e.g., dienogest or oxendolone), and 17α-spirolactone derviatives (e.g., drospirenone or spironolactone). SAAs may act as AR antagonists as well as antigonadotropins. Examples of non-steroidal antiandrogens (NSAAs) include first-generation NSAAs (e.g., bicalutamide, flutamide, or nilutamide), second-generation NSAAs (e.g., apalutamide, darolutamide, or enzalutamide), or non-generational NSAAs (e.g., cimetidine or topilutamide).

The term “androgen receptor” or “AR” refers to a ligand-activated transcriptional regulatory protein that mediates induction of a variety of biological effects through its interaction with androgens, which induces conformational changes of the receptor that affect receptor-protein interactions and receptor-DNA interactions. AR is mainly expressed in androgen target tissues, such as the prostate, skeletal muscle, liver, and central nervous system (CNS), with the highest expression level observed in the prostate, adrenal gland, and epididymis. AR can be activated by the binding of endogenous androgens, including testosterone and 5α-dihydrotestosterone (5α-DHT). Unbound AR is mainly located in the cytoplasm and associated with a complex of heat shock proteins (Hsps, e.g., Hsp70, Hsp90, Hsp56, and p23) through interactions with the AR ligand-binding domain. Upon agonist binding (e.g., binding of an androgen), AR goes through a series of conformational changes: the heat shock proteins dissociate from AR, and the transformed AR undergoes dimerization, phosphorylation, and translocation to the nucleus, which is mediated by the nuclear localization signal. The translocated AR then binds to the androgen response element (ARE), which is characterized by the six-nucleotide half-site consensus sequence 5′-TGTTCT-3′ spaced by three random nucleotides and is located in the promoter or enhancer region of AR gene targets. Recruitment of other transcription co-regulators (including co-activators and co-repressors) and transcriptional machinery further ensures the transactivation of AR-regulated gene expression. All of these processes are initiated by the ligand-induced conformational changes in the ligand-binding domain.

An “androgen receptor (AR) antagonist” or “AR inhibitor” refers to an agent that inhibits or reduces, directly or indirectly, at least one activity of an AR polypeptide, including, but not limited to, co-activator binding (e.g., ANPK, ARA24, ARA54, ARA70, ARA160, ARA267, ARIP3, BAG-1L, (3-catenin, BRCA1, Caveolin-1, BCP, Cyclin E, E6-Ap, FHL2, Gelsolin, HMG-1/-2, HSP40, PGC-1, PIAS1, RAF, Rb, RIP140, SNURF, SRC-1, SRC-3, Supervillin, TIF2, Tip60, Ubc9, and/or Zac-1 binding), co-repressor binding (e.g., calreticulin, Cyclin D1, and/or HBO1 binding), DNA binding (e.g., binding to the androgen response element of AR-regulated genes), ligand binding (e.g., an androgen, e.g., testosterone or 5α-DHT), or nuclear translocation. For a review of additional examples of AR co-regulators (e.g., co-activators, co-repressors), see, e.g., Heinlein et al., Endocrine Reviews. 23(2):175-200 (2002), which is incorporated herein by reference.

An “androgen synthesis inhibitor” refers to an agent that inhibits the enzymatic biosynthesis of androgens. Examples of androgen synthesis inhibitors include CYP17A1 inhibitors (e.g., abiraterone acetate, ketoconazole, or seviteronel), CYP11A1 (P450scc) inhibitors (e.g., aminoglutethimide), or 5α-reductase inhibitors (e.g., alfatradiol, dutasteride, or finasteride).

The term “anti-cancer therapy” refers to a therapy useful in treating cancer (e.g., prostate cancer, e.g., castration-resistant prostate cancer (CRPC), e.g., metastatic CRPC (mCRPC) or locally confined, inoperable CRPC). Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer. Combinations thereof are also included in the invention.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments.

The terms “anti-PD-L1 antibody” and “an antibody that binds to PD-L1” refer to an antibody that is capable of binding PD-L1 with sufficient affinity such that the antibody is useful as a diagnostic and/or therapeutic agent in targeting PD-L1. In one embodiment, the extent of binding of an anti-PD-L1 antibody to an unrelated, non-PD-L1 protein is less than about 10% of the binding of the antibody to PD-L1 as measured, e.g., by a radioimmunoassay (RIA). In certain embodiments, an anti-PD-L1 antibody binds to an epitope of PD-L1 that is conserved among PD-L1 from different species. In certain embodiments, the anti-PD-L1 antibody is atezolizumab. PD-L1 (programmed death ligand 1) is also referred to in the art as “programmed cell death 1 ligand 1,” “PDCD1LG1,” “CD274,” “B7-H,” and “PDL1.” An exemplary human PD-L1 is shown in UniProtKB/Swiss-Prot Accession No.Q9NZQ7.1.

An “article of manufacture” is any manufacture (e.g., a package or container) or kit comprising at least one reagent, e.g., a medicament for treatment of a disease or disorder (e.g., cancer, e.g., prostate cancer, e.g., CRPC, e.g., mCRPC or locally confined, inoperable CRPC), or a probe for specifically detecting a biomarker described herein. In certain embodiments, the manufacture or kit is promoted, distributed, or sold as a unit for performing the methods described herein.

A “blocking” antibody or an “antagonist” antibody is one which inhibits or reduces biological activity of the antigen it binds. Preferred blocking antibodies or antagonist antibodies substantially or completely inhibit the biological activity of the antigen.

By “binding domain” is meant a part of a compound or a molecule that specifically binds to a target epitope, antigen, ligand, or receptor. Binding domains include but are not limited to antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer, e.g., prostate cancer, e.g., CRPC, e.g., mCRPC or locally confined, inoperable CRPC) characterized by certain, molecular, pathological, histological, and/or clinical features. In some embodiments, a biomarker is a gene. Biomarkers include, but are not limited to, polynucleotides (e.g., DNA, and/or RNA), polynucleotide copy number alterations (e.g., DNA copy numbers), polypeptides, polypeptide and polynucleotide modifications (e.g., posttranslational modifications), carbohydrates, and/or glycolipid-based molecular markers.

The terms “cell proliferative disorder” and “proliferative disorder” refer to disorders that are associated with some degree of abnormal cell proliferation. In one embodiment, the cell proliferative disorder is cancer (e.g., prostate cancer, e.g., CRPC, e.g., mCRPC or locally confined, inoperable CRPC). In another embodiment, the cell proliferative disorder is a tumor.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include, but not limited to, prostate cancer, such as castration-resistant prostate cancer (CRPC), which includes metastatic CRPC (mCRPC) and locally confined, inoperable CRPC; squamous cell cancer (e.g., epithelial squamous cell cancer); lung cancer, including small-cell lung cancer, non-small cell lung cancer, adenocarcinoma of the lung, and squamous carcinoma of the lung; cancer of the peritoneum; hepatocellular cancer; gastric or stomach cancer, including gastrointestinal cancer and gastrointestinal stromal cancer; pancreatic cancer; glioblastoma; cervical cancer; ovarian cancer; liver cancer; bladder cancer (e.g., urothelial bladder cancer (UBC), muscle invasive bladder cancer (MIBC), and BCG-refractory non-muscle invasive bladder cancer (NMIBC)); cancer of the urinary tract; hepatoma; breast cancer (e.g., HER2+breast cancer and triple-negative breast cancer (TNBC), which are estrogen receptors (ER−), progesterone receptors (PR−), and HER2 (HER2-) negative); colon cancer; rectal cancer; colorectal cancer; endometrial or uterine carcinoma; salivary gland carcinoma; kidney or renal cancer (e.g., renal cell carcinoma (RCC)); prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

A “chemotherapeutic agent” is a chemical compound useful in the treatment of cancer (e.g., prostate cancer, e.g., CRPC, e.g., mCRPC or locally confined, inoperable CRPC). Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1¹ and calicheamicin omegall (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton, N.J.), albumin-engineered nanoparticle formulation of paclitaxel (ABRAXANE™), and docetaxel (TAXOTERE®, Rhome-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g., celecoxib or etoricoxib), proteosome inhibitor (e.g., PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors; tyrosine kinase inhibitors; serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin, and pharmaceutically acceptable salts, acids or derivatives of any of the above; as well as combinations of two or more of the above.

Chemotherapeutic agents as defined herein include “anti-hormonal agents” or “endocrine therapeutics” which act to regulate, reduce, block, or inhibit the effects of hormones that can promote the growth of cancer (e.g., prostate cancer, e.g., CRPC, e.g., mCRPC or locally confined, inoperable CRPC). They may be hormones themselves, including, but not limited to: anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON.cndot.toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; Vinorelbine and Esperamicins (see U.S. Pat. No. 4,675,187), and pharmaceutically acceptable salts, acids or derivatives of any of the above; as well as combinations of two or more of the above.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgGi, IgG2, IgG3, IgG4, IgAi, and IgA2. The heavy chain constant domains that correspond to the different classes of immunoglobulins are called α, δ, ε, γ, and μ, respectively.

It is understood that aspects and embodiments of the invention described herein include “comprising,” “consisting,” and “consisting essentially of” aspects and embodiments.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

As used herein, “delaying progression” of a disorder or disease means to defer, hinder, slow, retard, stabilize, and/or postpone development of the disease or disorder (e.g., cancer, e.g., prostate cancer, e.g., CRPC, e.g., mCRPC or locally confined, inoperable CRPC). This delay can be of varying lengths of time, depending on the history of the disease and/or subject being treated. As is evident to one skilled in the art, a sufficient or significant delay can, in effect, encompass prevention, in that the subject does not develop the disease.

The term “detection” includes any means of detecting, including direct and indirect detection.

A “disorder” or “disease” is any condition that would benefit from treatment including, but not limited to, chronic and acute disorders or diseases including those pathological conditions which predispose the mammal to the disorder in question (e.g., cancer, e.g., prostate cancer, e.g., CRPC, e.g., mCRPC or locally confined, inoperable CRPC).

“Effector functions” refer to those biological activities attributable to the Fc region of an antibody, which vary with the antibody isotype. Examples of antibody effector functions include: Cl q binding and complement dependent cytotoxicity (CDC); Fc receptor binding; antibody-dependent cell-mediated cytotoxicity (ADCC); phagocytosis; down regulation of cell surface receptors (e.g., PD-L1); and B cell activation.

An “effective amount” of a compound, for example, an anti-PD-L1 antibody or antiandrogen, or a composition (e.g., pharmaceutical composition) thereof, is at least the minimum amount required to achieve the desired therapeutic or prophylactic result, such as a measurable increase in overall survival or progression-free survival of a particular disease or disorder (e.g., a cancer, e.g., prostate cancer, e.g., CRPC, e.g., mCRPC or locally confined, inoperable CRPC). An effective amount herein may vary according to factors such as the disease state, age, sex, and weight of the patient, and the ability of the antibody to elicit a desired response in the subject. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease (e.g., reduction or delay in cancer-related pain, symptomatic skeletal-related events (SSE), reduction in symptoms per the European Organization for Research and Treatment of Cancer Quality-of-Life Questionnaire (EORTC QLQ-C30, e.g., fatigue, nausea, vomiting, pain, dyspnea, insomnia, appetite loss, constipation, diarrhea, or general level of physical emotional, cognitive, or social functioning), reduction in pain as measured by, e.g., the 10-point pain severity (measured at its worst) numerical rating scale (NRS), and/or reduction in urinary symptoms associated with prostate cancer per the EORTC Quality-of-Life Questionnaire—Urinary Scale (QLQ-PR25, e.g., increased urination frequency, urination pain, or incontinence), increasing the quality of life of those suffering from the disease, decreasing the dose of other medications required to treat the disease, enhancing effect of another medication such as via targeting, delaying the progression of the disease (e.g. progression-free survival or radiographic progression-free survival (rPFS); delay of unequivocal clinical progression (e.g., cancer-related pain progression, symptomatic skeletal-related event, deterioration in Eastern Cooperative Group Oncology Group (ECOG) performance status (e.g., how the disease affects the daily living abilities of the patient), and/or initiation of next systemic anti-cancer therapy), and/or delaying time to prostate-specific antigen progression), and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

“Elevated expression,” “elevated expression levels,” or “elevated levels” refers to an increased expression or increased levels of a biomarker in a subject relative to a control, such as a subject or subjects who are not suffering from the disease or disorder (e.g., cancer, e.g., prostate cancer, e.g., CRPC, e.g., mCRPC or locally confined, inoperable CRPC) or an internal control (e.g., housekeeping biomarker).

A subject who has “failed to respond to” or “failed” a treatment refers to an individual who displays disease progression (e.g., progression of a cancer, e.g., prostate cancer, e.g., CRPC, e.g., mCRPC or locally confined, inoperable CRPC) following treatment (e.g., after receiving treatment with an androgen synthesis inhibitor for at least 28 days).

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full-length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Individual response” or “response” can be assessed using any endpoint indicating a benefit to the subject, including, without limitation, (1) inhibition, to some extent, of disease progression (e.g., progression of cancer, e.g., prostate cancer, e.g., CRPC, e.g., mCRPC or locally confined, inoperable CRPC), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e. reduction, slowing down or complete stopping) of metastasis; (5) relief, to some extent, of one or more symptoms associated with the disease or disorder (e.g., cancer, e.g., prostate cancer, e.g., CRPC, e.g., mCRPC or locally confined, inoperable CRPC); (6) increase or extend in the length of survival, including overall survival and progression-free survival; and/or (9) decreased mortality at a given point of time following treatment.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The terms “level of expression” or “expression level” in general are used interchangeably and generally refer to the amount of a biomarker in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

“Prostate cancer” refers to an adenocarcinoma of the prostate, which may have been histologically confirmed. The prostate cancer may be either metastatic or non-metastatic. In some instances, the prostate cancer is locally confined, inoperable prostate cancer that cannot be treated with definitive intent (e.g., no chance for curative intervention).

“Castration-resistant prostate cancer” or “CRPC,” as used herein, is a prostate cancer defined by disease progression, as measured by prostate-specific antigen (PSA) or radiographic measures, despite adequate suppression of testosterone levels (e.g., castrate serum testosterone level is less than or equal to 50 ng/dl (1.7 nmol/L)). A subject having CRPC may, for example, show disease progression of prostate cancer after having undergone surgical castration (e.g., bilateral orchiectomy) or chemical castration (e.g., maintenance on androgen ablation therapy with luteinizing hormone-releasing hormone agonist or antagonist or polyestradiol phosphate). The CRPC may be either metastatic (mCRPC) or locally confined, inoperable CRPC.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “PD-1 axis binding antagonist” refers to a molecule that inhibits the interaction of a PD-1 axis binding partner with either one or more of its binding partner, so as to remove T-cell dysfunction resulting from signaling on the PD-1 signaling axis, with a result being to restore or enhance T-cell function (e.g., proliferation, cytokine production, target cell killing). As used herein, a PD-1 axis binding antagonist includes a PD-1 binding antagonist, a PD-L1 binding antagonist, and a PD-L2 binding antagonist.

The term “PD-1 binding antagonist” refers to a molecule that decreases, blocks, inhibits, abrogates, or interferes with signal transduction resulting from the interaction of PD-1 with one or more of its binding partners, such as PD-L1, PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate, or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-L1 antibody.

The term “PD-L1 binding antagonist” refers to a molecule that decreases, blocks, inhibits, abrogates, or interferes with signal transduction resulting from the interaction of PD-L1 with either one or more of its binding partners, such as PD-1 or B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen-binding fragments thereof, immunoadhesins, fusion proteins, oligopeptides, and other molecules that decrease, block, inhibit, abrogate, or interfere with signal transduction resulting from the interaction of PD-L1 with one or more of its binding partners, such as PD-1 or B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific embodiment, the anti-PD-L1 antibody is atezolizumab (CAS Registry Number: 1422185-06-5), also known as MPDL3280A, and described herein. In another specific embodiment, the anti-PD-L1 antibody is YW243.55.S70, described herein. In another specific embodiment, the anti-PD-L1 antibody is MDX-1105, described herein. In still another specific aspect, the anti-PD-L1 antibody is MED14736, described herein.

As used herein, “complete response” or “CR” refers to disappearance of all target lesions.

As used herein, “partial response” or “PR” refers to at least a 30% decrease in the sum of the longest diameters (SLD) of target lesions, taking as reference the baseline SLD.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression-free survival” (PFS) refers to the length of time during and after treatment during which the disease being treated (e.g., cancer, e.g., prostate cancer, e.g., CRPC, e.g., mCRPC or local confined, inoperable CRPC) does not get worse. Progression-free survival may include the amount of time patients have experienced a complete response or a partial response, as well as the amount of time patients have experienced stable disease.

As used herein, “overall survival” (OS) refers to the percentage of subjects in a group who are likely to be alive after a particular duration of time.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated (e.g., cancer, e.g., prostate cancer, e.g., CRPC, e.g., mCRPC or locally confined, inoperable CRPC), the presence or size of metastases, or the size of the primary tumor.

“Reduced expression,” “reduced expression levels,” or “reduced levels” refers to a decrease expression or decreased levels of a biomarker in a subject relative to a control, such as a subject or subjects who are not suffering from the disease or disorder (e.g., cancer, e.g., prostate cancer, e.g., CRPC, e.g., mCRPC or locally confined, inoperable CRPC) or an internal control (e.g., housekeeping biomarker). In some embodiments, reduced expression is little or no expression.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject. In yet another embodiment, a reference sample, reference cell, reference tissue, control sample, control cell, or control tissue is obtained from a healthy and/or non-diseased part of the body (e.g., tissues or cells) of a subject who is not the subject. In even another embodiment, a reference sample, reference cell, reference tissue, control sample, control cell, or control tissue is obtained from an untreated tissue and/or cell of the body of an individual who is not the subject.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

A “subject” or an “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the subject or individual is a human.

As used herein, “treatment” (and grammatical variations thereof, such as “treat” or “treating”) refers to clinical intervention in an attempt to alter the natural course of the subject being treated, and can be performed either for prophylaxis or during the course of clinical pathology. Desirable effects of treatment include, but are not limited to, preventing occurrence or recurrence of disease (e.g., prostate cancer, e.g., CRPC, e.g., mCRPC or locally confined, inoperable CRPC), alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease (e.g., prostate cancer, e.g., CRPC, e.g., mCRPC or locally confined, inoperable CRPC). In some instances, the treatment may increase overall survival (OS) (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater). In some instances, the treatment may increase the progression-free survival (PFS) (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater).

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease (e.g., prostate cancer, e.g., CRPC, e.g., mCRPC or locally confined, inoperable CRPC) tissue/organ. The tissue sample may contain compounds which are not naturally intermixed with the tissue in nature such as preservatives, anticoagulants, buffers, fixatives, nutrients, antibiotics, or the like.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### II. Methods of Treatment

Provided herein are methods for treating or delaying progression of cancer (e.g., a prostate cancer, e.g., a castration-resistant prostate cancer (CRPC), e.g., metastatic CRPC (mCRPC) or locally confined, inoperable CRPC)) in a subject comprising administering to the subject an effective amount of a PD-1 axis binding antagonist (e.g., PD-L1 binding antagonist, e.g., anti-PD-L1 antibody, e.g., atezolizumab) and an antiandrogen (e.g., an androgen receptor (AR) antagonist, e.g., enzalutamide).

A. Dosing and Administration

The methods of the invention described herein include administering a therapeutically effective amount of a PD-1 axis binding antagonist (e.g., PD-L1 binding antagonist, e.g., anti-PD-L1 antibody, e.g., atezolizumab) and an antiandrogen (e.g., an AR antagonist, e.g., enzalutamide) to a subject having a cancer (e.g., prostate cancer, e.g., CRPC, e.g., mCRPC or locally confined, inoperable CRPC), thereby treating the subject. In particular instances, the subject has metastatic CRPC (mCRPC) and has previously failed treatment with an androgen synthesis inhibitor and has failed, is ineligible for, or has refused a taxane regimen. The appropriate doses and dosing regimen for the PD-1 axis binding antagonist (e.g., PD-L1 binding antagonist, e.g., anti-PD-L1 antibody, e.g., atezolizumab) and/or the antiandrogen (e.g., an AR antagonist, e.g., enzalutamide) may be determined based on the severity and course of the disease (e.g., prostate cancer, e.g., CRPC, e.g., mCRPC or locally confined, inoperable CRPC), the clinical condition of the subject, the subject's clinical history and response to the treatment, and the discretion of the attending physician.

In some instances, the therapeutically effective amount of the PD-1 axis binding antagonist (e.g., PD-L1 binding antagonist, e.g., anti-PD-L1 antibody, e.g., atezolizumab) may be between about 60 mg to about 5000 mg (e.g., between about 60 mg to about 4500 mg, between about 60 mg to about 4000 mg, between about 60 mg to about 3500 mg, between about 60 mg to about 3000 mg, between about 60 mg to about 2500 mg, between about 650 mg to about 2000 mg, between about 60 mg to about 1500 mg, between about 100 mg to about 1500 mg, between about 300 mg to about 1500 mg, between about 500 mg to about 1500 mg, between about 700 mg to about 1500 mg, between about 1000 mg to about 1500 mg, between about 1000 mg to about 1400 mg, between about 1100 mg to about 1300 mg, between about 1150 mg to about 1250 mg, between about 1175 mg to about 1225 mg, or between about 1190 mg to about 1210 mg, e.g., about 1200 mg±5 mg, about 1200±2.5 mg, about 1200±1.0 mg, about 1200±0.5 mg, about 1200±0.2 mg, or about 1200±0.1 mg). In some instances, the therapeutically effective amount of the PD-1 axis binding antagonist (e.g., PD-L1 binding antagonist, e.g., anti-PD-L1 antibody, e.g., atezolizumab) may be between about 800 mg to about 1200 mg (e.g., between about 800 mg to between 1100 mg, between about 800 mg to about 1000 mg, between about 800 mg to about 900 mg, between about 800 mg to about 850 mg, between about 800 to about 825 mg, between about 800 mg to about 1200 mg, between about 850 mg to about 1200 mg, between about 900 mg to about 1200 mg, between about 950 mg to about 1200 mg, between about 1000 mg to about 1200 mg, between about 1050 mg to about 1200 mg, between about 1100 mg to about 1200 mg, between about 1125 mg to about 1200 mg, between about 1150 mg to about 1200 mg, or between about 1175 mg to about 1200 mg). In some instances, the PD-1 axis binding antagonist (e.g., PD-L1 binding antagonist, e.g., anti-PD-L1 antibody, e.g., atezolizumab) is administered at about 1200 mg (e.g., a fixed dose of about 1200 mg or about 15 mg/kg). In some instances, the amount of the PD-1 axis binding antagonist (e.g., PD-L1 binding antagonist, e.g., anti-PD-L1 antibody, e.g., atezolizumab) administered may be in the range of about 0.01 to about 50 mg/kg of the subject's body weight (e.g., between about 0.01 to about 45 mg/kg, between about 0.01 mg/kg to about 40 mg/kg, between about 0.01 mg/kg to about 35 mg/kg, between about 0.01 mg/kg to about 30 mg/kg, between about 0.1 mg/kg to about 30 mg/kg, between about 1 mg/kg to about 30 mg/kg, between about 2 mg/kg to about 30 mg/kg, between about 5 mg/kg to about 30 mg/kg, between about 5 mg/kg to about 25 mg/kg, between about 5 mg/kg to about 20 mg/kg, between about 10 mg/kg to about 20 mg/kg, or between about 12 mg/kg to about 18 mg/kg, e.g., about 15±2 mg/kg, about 15±1 mg/kg, about 15±0.5 mg/kg, about 15±0.2 mg/kg, or about 15±0.1 mg/kg). In some instances, the amount of the PD-1 axis binding antagonist (e.g., PD-L1 binding antagonist, e.g., anti-PD-L1 antibody, e.g., atezolizumab) administered may be in the range of about 10 mg/kg to about 15 mg/kg of the subject's body weight (e.g., between about 10 mg/kg to about 14 mg/kg, between about 10 mg/kg to about 13 mg/kg, between about 10 mg/kg to about 12 mg/kg, between about 10 mg/kg to about 11 mg/kg, between about 11 mg/kg to about 15 mg/kg, between about 12 mg/kg to about 15 mg/kg, or between about 13 mg/kg to about 15 mg/kg, e.g., about 15±1 mg/kg, about 15±0.5 mg/kg, about 15±0.2 mg/kg, or about 15±0.1 mg/kg). In some instances, the method includes administering the PD-1 axis binding antagonist (e.g., PD-L1 binding antagonist, e.g., anti-PD-L1 antibody, e.g., atezolizumab) at 15 mg/kg. In any dosage amount or formulation, the PD-1 axis binding antagonist (e.g., PD-L1 binding antagonist, e.g., anti-PD-L1 antibody, e.g., atezolizumab) may be administered as a single dose or as multiple doses (e.g., two or three doses). The dose of the antibody administered in a combination treatment may be reduced as compared to a single treatment. The progress of this therapy may be monitored by conventional techniques.

The methods of the invention further include administering a therapeutically effective amount of an antiandrogen (e.g., an AR antagonist, e.g., enzalutamide). In some instances, the therapeutically effective amount of the antiandrogen (e.g., an AR antagonist, e.g., enzalutamide) may be between about 1 mg to about 600 mg (e.g., between about 1 mg to about 550 mg, between about 1 mg to about 500 mg, between about 1 mg to about 450 mg, between about 1 mg to about 400 mg, between about 1 mg to about 350 mg, between about 1 mg to about 300 mg, between about 10 mg to about 300 mg, between about 20 mg to about 300 mg, between about 40 mg to about 300 mg, between about 50 mg to about 300 mg, between about 50 mg to about 250 mg, between about 75 mg to about 225 mg, between about 100 mg to about 200 mg, between about 110 mg to about 190 mg, between about 120 mg to about 180 mg, between about 130 mg to about 190 mg, between about 140 mg to about 180 mg, between about 150 mg to about 170 mg, or between about 155 mg to about 165 mg, e.g., about 160 mg±2.5 mg, about 160 mg±1 mg, about 160±0.5 mg, about 160±0.2 mg, or about 160±0.1 mg). In some instances, the therapeutically effective amount of the antiandrogen (e.g., an AR antagonist, e.g., enzalutamide) may be between about 80 mg to about 240 mg (e.g., between about 80 mg to about 220 mg, between about 80 mg to about 200 mg, between about 80 mg to about 160 mg, between about 80 mg to about 100 mg, between about 100 mg to about 200 mg, between about 120 mg to about 180 mg, between about 140 mg to about 170 mg, between about 150 mg to about 170 mg, or between about 155 mg to about 165 mg, e.g., about 160 mg±2.5 mg, about 160 mg±1 mg, about 160±0.5 mg, about 160±0.2 mg, or about 160±0.1 mg). In some instances, the method includes administering the antiandrogen (e.g., an AR antagonist, e.g., enzalutamide) at a dose of about 160 mg. In any dosage amount or formulation, the antiandrogen (e.g., an AR antagonist, e.g., enzalutamide) may be administered as a single dose or as multiple doses (e.g., two or three doses). In some instances, the antiandrogen (e.g., an AR antagonist, e.g., enzalutamide) may be administered in two or more doses. In some instances, the antiandrogen (e.g., an AR antagonist, e.g., enzalutamide) is administered in four doses of about 40 mg per dose.

In any of the methods described herein, the PD-1 axis binding antagonist (e.g., PD-L1 binding antagonist, e.g., anti-PD-L1 antibody, e.g., atezolizumab) and the antiandrogen (e.g., an AR antagonist, e.g., enzalutamide) may be administered in one or more dosing cycles (e.g., 1, 2, 3, 4, 5, 6, 7, 8, 9, or 10 or more dosing cycles). In some instances, the length of each dosing cycle may be about 18 to 24 days (e.g., 15 days, 16 days, 17 days, 18 days, 19 days, 20 days, 21 days, 22 days, 23 days, or 24 days). In some instances, the length of the dosing cycle may be about 21 days. In some instances, the PD-1 axis binding antagonist (e.g., PD-L1 binding antagonist, e.g., anti-PD-L1 antibody, e.g., atezolizumab) may be administered on about Day 1 of the dosing cycle (e.g., Day 1+/−3 days). For example, the PD-1 axis binding antagonist (e.g., PD-L1 binding antagonist, e.g., anti-PD-L1 antibody, e.g., atezolizumab) may be administered intravenously at a dose of 1200 mg on Day 1 of each 21-day cycle. In some instances, the antiandrogen (e.g., an AR antagonist, e.g., enzalutamide) may be administered in one or more doses each day of the dosing cycle. For example, the antiandrogen (e.g., an AR antagonist, e.g., enzalutamide) may be administered orally at a dose of 160 mg (e.g., four 40-mg capsules) daily. In some instances, the subject receives treatment with a PD-1 axis binding antagonist (e.g., PD-L1 binding antagonist, e.g., anti-PD-L1 antibody, e.g., atezolizumab) and an antiandrogen (e.g., an AR antagonist, e.g., enzalutamide) in one or more dosing cycles until loss of clinical benefit (e.g., confirmed disease progression, drug resistance, death, or unacceptable toxicity).

In any of the methods described herein, the PD-1 axis binding antagonist (e.g., PD-L1 binding antagonist, e.g., anti-PD-L1 antibody, e.g., atezolizumab) and/or the antiandrogen (e.g., an AR antagonist, e.g., enzalutamide) may be administered in any suitable manner known in the art. For example, the PD-1 axis binding antagonist (e.g., PD-L1 binding antagonist, e.g., anti-PD-L1 antibody, e.g., atezolizumab) and the antiandrogen (e.g., an AR antagonist, e.g., enzalutamide) may be administered sequentially (at different times) or concurrently (at about the same time). In some instances, the PD-1 axis binding antagonist (e.g., PD-L1 binding antagonist, e.g., anti-PD-L1 antibody, e.g., atezolizumab) and the antiandrogen (e.g., an AR antagonist, e.g., enzalutamide) are administered during the same dosing cycle, but with different dosing regimens (e.g., administered on different days and/or administered at different frequencies). In some embodiments, the PD-1 axis binding antagonist (e.g., PD-L1 binding antagonist, e.g., anti-PD-L1 antibody, e.g., atezolizumab) is in a separate composition as the antiandrogen (e.g., an AR antagonist, e.g., enzalutamide). In some embodiments, the PD-1 axis binding antagonist (e.g., PD-L1 binding antagonist, e.g., anti-PD-L1 antibody, e.g., atezolizumab) is in the same composition as the antiandrogen (e.g., an AR antagonist, e.g., enzalutamide).

Further, the PD-1 axis binding antagonist (e.g., PD-L1 binding antagonist, e.g., anti-PD-L1 antibody, e.g., atezolizumab) and the antiandrogen (e.g., an AR antagonist, e.g., enzalutamide) may be administered by the same route of administration or by different routes of administration. In some instances, the PD-1 axis binding antagonist (e.g., PD-L1 binding antagonist, e.g., anti-PD-L1 antibody, e.g., atezolizumab) can be administered intravenously, intramuscularly, subcutaneously, topically, orally, transdermally, intraperitoneally, intraorbitally, by implantation, by inhalation, intrathecally, intraventricularly, or intranasally. In one particular instance, the PD-1 axis binding antagonist (e.g., PD-L1 binding antagonist, e.g., anti-PD-L1 antibody, e.g., atezolizumab) can be administered intravenously (e.g., intravenous infusion). In some instances, the antiandrogen (e.g., an AR antagonist, e.g., enzalutamide) can be administered orally, intravenously, intramuscularly, subcutaneously, topically, transdermally, intraperitoneally, intraorbitally, by implantation, by inhalation, intrathecally, intraventricularly, or intranasally. In one particular instance, the antiandrogen (e.g., an AR antagonist, e.g., enzalutamide) can be administered orally.

In some instances, the methods include administering to the subject atezolizumab at a dose of about 1200 mg intravenously on the first day of each dosing cycle (e.g., 21-day dosing cycle) and enzalutamide at a dose of about 160 mg (e.g., four doses at about 40 mg per dose) orally on each day of each dosing cycle (e.g., 21-day dosing cycle).

B. PD-1 Axis Binding Antagonist

Provided herein are methods for treating or delaying progression of cancer (e.g., prostate cancer, e.g., CRPC, e.g., mCRPC or locally confined, inoperable CRPC) in a subject comprising administering to the subject an effective amount of a PD-1 axis binding antagonist (e.g., a PD-L1 binding antagonist, e.g., anti-PD-L1 antibody, e.g., atezolizumab) and an antiandrogen (e.g., an AR antagonist, e.g., enzalutamide). The PD-1 axis binding antagonist may, in some instances, be a PD-1 binding antagonist, a PD-L1 binding antagonist, or a PD-L2 binding antagonist.

In some instances, the PD-L1 binding antagonist is an anti-PD-L1 antibody. PD-L1 (programmed death ligand 1), also known as PD-L1, B7-H1, B7-4, CD274, and B7-H, is a transmembrane protein, and its interaction with PD-1 inhibits T-cell activation and cytokine production. In some instances, the anti-PD-L1 antibody inhibits the binding of PD-L1 to its binding partners. In a specific aspect, PD-L1 binding partners are PD-1 and/or B7-1. In some instances, the anti-PD-L1 antibody is capable of inhibiting binding between PD-L1 and PD-1 and/or between PD-L1 and B7-1.

In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)₂ fragments. In some instances, the anti-PD-L1 antibody is a humanized antibody. In some instances, the anti-PD-L1 antibody is a human antibody. In some instances, the anti-PD-L1 antibody described herein binds to human PD-L1.

In some particular instances, the anti-PD-L1 antibody is atezolizumab (CAS Registry Number: 1422185-06-5). Atezolizumab (Genentech) is also known as MPDL3280A.

Atezolizumab comprises a heavy chain variable region (HVR-H) comprising an HVR-H1, HVR-H2, and HVR-H3 sequence, wherein:

(a) the HVR-H1 sequence is
(SEQ ID NO: 1)
GFTFSDSWIH;
(b) the HVR-H2 sequence is
(SEQ ID NO: 2)
AWISPYGGSTYYADSVKG;
and
(c) the HVR-H3 sequence is
(SEQ ID NO: 3)
RHWPGGFDY.

Atezolizumab further comprises a light chain variable region (HVR-L) comprising an HVR-L1, HVR-L2, and HVR-L3 sequence, wherein:

(a) the HVR-L1 sequence is
(SEQ ID NO: 4)
RASQDVSTAVA;
(b) the HVR-L2 sequence is
(SEQ ID NO: 5)
SASFLYS;
and
(c) the HVR-L3 sequence is
(SEQ ID NO: 6)
QQYLYHPAT.

Atezolizumab comprises a heavy chain and a light chain sequence, wherein:

(a) the heavy chain variable (VH) region sequence comprises the amino acid sequence:

(SEQ ID NO: 7)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS;

and

(b) the light chain variable (VL) region sequence comprises the amino acid sequence:

(SEQ ID NO: 8)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

Atezolizumab comprises a heavy chain and a light chain sequence, wherein:

(a) the heavy chain comprises the amino acid sequence:

(SEQ ID NO: 9)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG;

and

(b) the light chain comprises the amino acid sequence:

(SEQ ID NO: 10)
DIQMTOSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some instances, the anti-PD-L1 antibody comprises (a) a VH domain comprising an amino acid sequence comprising having at least 95% sequence identity (e.g., at least 95%, 96%, 97%, 98%, or 99% sequence identity) to, or the sequence of (SEQ ID NO: 7); (b) a VL domain comprising an amino acid sequence comprising having at least 95% sequence identity (e.g., at least 95%, 96%, 97%, 98%, or 99% sequence identity) to, or the sequence of (SEQ ID NO: 8); or (c) a VH domain as in (a) and a VL domain as in (b). In other instances, the anti-PD-L1 antibody is selected from YW243.55.570, MDX-1105, and MED14736 (durvalumab), and MSB0010718C (avelumab). Antibody YW243.55.570 is an anti-PD-L1 described in PCT Pub. No. WO 2010/077634. MDX-1105, also known as BMS-936559, is an anti-PD-L1 antibody described in PCT Pub. No. WO 2007/005874. MED14736 (durvalumab) is an anti-PD-L1 monoclonal antibody described in PCT Pub. No. WO 2011/066389 and U.S. Pub. No. 2013/034559. Examples of anti-PD-L1 antibodies useful for the methods of this invention, and methods for making thereof are described in PCT Pub. Nos. WO 2010/077634, WO 2007/005874, and WO 2011/066389, and also in U.S. Pat. No. 8,217,149, and U.S. Pub. No. 2013/034559, which are incorporated herein by reference. The PD-1 axis binding antagonists (e.g., PD-L1 binding antagonist, e.g., anti-PD-L1 antibody, e.g., atezolizumab) useful in this invention, including compositions containing such antibodies, may be used in combination with an antiandrogen (e.g., an AR antagonist, e.g., enzalutamide) to treat cancer (e.g., prostate cancer, e.g., CRPC, e.g., mCRPC or locally confined, inoperable CRPC).

In some instances, the PD-1 binding antagonist is an anti-PD-1 antibody, such as an anti-PD-1 antibody selected from the group consisting of MDX-1106 (nivolumab), MK-3475 (pembrolizumab), CT-011 (pidilizumab), MEDI-0680 (AMP-514), PDR001, REGN2810, and BGB-108. MDX-1106, also known as MDX-1106-04, ONO-4538, BMS-936558, or nivolumab, is an anti-PD-1 antibody described in PCT Pub. No. WO 2006/121168. MK-3475, also known as pembrolizumab or lambrolizumab, is an anti-PD-1 antibody described in PCT Pub. No. WO 2009/114335. CT-011, also known as hBAT, hBAT-1 or pidilizumab, is an anti-PD-1 antibody described in PCT Pub. No. WO 2009/101611. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In other instances, the PD-1 binding antagonist is AMP-224. AMP-224, also known as B7-DCIg, is a PD-L2-Fc fusion soluble receptor described in PCT Pub. Nos. WO 2010/027827 and WO 2011/066342.

In other instances, the PD-L2 binding antagonist is an anti-PD-L2 antibody (e.g., a human, a humanized, or a chimeric anti-PD-L2 antibody). In some instances, the PD-L2 binding antagonist is an immunoadhesin.

(i) Substitution, Insertion, and Deletion Variants

In certain instances, PD-1 axis binding antagonist (e.g., PD-L1 binding antagonist, e.g., anti-PD-L1 antibody, e.g., atezolizumab) variants having one or more amino acid substitutions are provided for use in the methods, compositions, and/or kits of the invention. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 1
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

(ii) Glycosylation Variants

In some instances, the PD-1 axis binding antagonist (e.g., PD-L1 binding antagonist, e.g., anti-PD-L1 antibody, e.g., atezolizumab) variant has been modified to increase or decrease the extent to which the bispecific antibody is glycosylated. Addition or deletion of glycosylation sites to a PD-1 axis binding antagonist (e.g., PD-L1 binding antagonist, e.g., anti-PD-L1 antibody, e.g., atezolizumab) may be conveniently accomplished by altering the amino acid sequence such that one or more glycosylation sites is created or removed.

Where the bispecific antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In some instances, the PD-1 axis binding antagonist (e.g., PD-L1 binding antagonists, e.g., anti-PD-L1 antibody, e.g., atezolizumab) variant has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO 2003/085107).

In view of the above, in some instances, the methods of the invention involve administering to the subject in the context of a fractionated, dose-escalation dosing regimen a PD-1 axis binding antagonist (e.g., PD-L1 binding antagonist, e.g., anti-PD-L1 antibody, e.g., atezolizumab) variant that comprises an aglycosylation site mutation. In some instances, the aglycosylation site mutation reduces effector function of the bispecific antibody. In some instances, the aglycosylation site mutation is a substitution mutation. In some instances, the bispecific antibody comprises a substitution mutation in the Fc region that reduces effector function. In some instances, the substitution mutation is at amino acid residue N297, L234, L235, and/or D265 (EU numbering). In some instances, the substitution mutation is selected from the group consisting of N297G, N297A, L234A, L235A, D265A, and P329G. In some instances, the substitution mutation is at amino acid residue N297. In a preferred embodiment, the substitution mutation is N297A.

In other instances, bispecific antibody variants with bisected oligosaccharides are used in accordance with the methods of the invention, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

(iii) Fc Region Variants

In some instances, a PD-1 axis binding antagonist (e.g., PD-L1 binding antagonist, e.g., anti-PD-L1 antibody, e.g., atezolizumab) variant that has one or more amino acid modifications introduced into the Fc region (i.e., an Fc region variant (see e.g., US 2012/0251531)) of the bispecific antibody may be administered to a subject having cancer (e.g., prostate cancer, e.g., CRPC, e.g., mCRPC or locally confined, inoperable CRPC) in accordance with the methods of the invention. The Fc region variant may comprise a human Fc region sequence (e.g., a human IgG1, IgG2, IgG3 or IgG4 Fc region) comprising an amino acid modification (e.g., a substitution) at one or more amino acid positions.

In some instances, the bispecific Fc region antibody variant possesses some but not all effector functions, which makes it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc RIII only, whereas monocytes express Fc RI, Fc RII and Fc RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CYTOTOX96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain instances, the proline at position 329 of a wild-type human Fc region in the antibody is substituted with glycine or arginine or an amino acid residue large enough to destroy the proline sandwich within the Fc/Fcγ receptor interface that is formed between the proline 329 of the Fc and tryptophan residues Trp 87 and Trp 110 of FcgRIII (Sondermann et al. Nature. 406, 267-273 (2000)). In certain embodiments, the bispecific antibody comprises at least one further amino acid substitution. In one embodiment, the further amino acid substitution is S228P, E233P, L234A, L235A, L235E, N297A, N297D, or P331S, and still in another embodiment the at least one further amino acid substitution is L234A and L235A of the human IgG1 Fc region or S228P and L235E of the human IgG4 Fc region (see e.g., US 2012/0251531), and still in another embodiment the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain instances, the PD-1 axis binding antagonist (e.g., PD-L1 binding antagonist, e.g., anti-PD-L1 antibody, e.g., atezolizumab) comprises an Fc region with one or more amino acid substitutions which improve ADCC, e.g., substitutions at positions 298, 333, and/or 334 of the Fc region (EU numbering of residues).

In some instances, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

(iv) Cysteine Engineered Antibody Variants

In certain embodiments, it may be desirable to create cysteine engineered anti-PD-L1 antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541

(v) Other Antibody Derivatives

In some instances, the PD-1 axis binding antagonist (e.g., PD-L1 binding antagonist, e.g., anti-PD-L1 antibody, e.g., atezolizumab) may be modified to contain additional non-proteinaceous moieties that are known in the art and readily available and administered to the subject in accordance with the methods described herein. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

C. Antiandrogens

Antiandrogens for use in the methods and/or compositions (e.g., pharmaceutical compositions, kits, etc.) of the invention may, in some instances, be AR antagonists (e.g., enzalutamide). The AR antagonists may be steroidal or non-steroidal AR antagonists. In some instances, the AR antagonists may include a non-steroidal antiandrogen (NSAA) including, but not limited to, first-generation NSAAs (e.g., bicalutamide, flutamide, or nilutamide), second-generation NSAAs (e.g., apalutamide, darolutamide, or enzalutamide), or non-generational NSAAs (e.g., cimetidine or topilutamide). In some instances, the NSAA is enzalutamide (e.g., 4-(3-(4-Cyano-3-(trifluoromethyl)phenyl)-5,5-dimethyl-4-oxo-2-thioxoimidazolidin-1-yl)-2-fluoro-N-methylbenzamide, XTANDI® (Medivation, Astellas)), or a pharmaceutically acceptable salt thereof. Exemplary methods for the administration of enzalutamide (XTANDI®) are described in Prescribing Information for enzalutamide (XTANDI®) in the United States, Astellas Pharma US, Inc. (Oct. 20, 2016), which is incorporated herein by reference in its entirety.

In some instances, the AR antagonist may include a steroidal antiandrogen (SAA) including, but are not limited to, 17α-hydroxyprogesterone derivatives (e.g., chlormadinone acetate, cyproterone acetate, or megestrol acetate), 19-nortestosterone derivatives (e.g., dienogest or oxendolone), and 17α-spirolactone derivatives (e.g., drospirenone or spironolactone). In some instances, the SAA may act as an AR antagonist and an antigonadotropin.

D. Treatment Indications

The methods of the invention described herein may be useful for treating a subject who has cancer (e.g., prostate cancer, e.g., CRPC, e.g., mCRPC or locally confined, inoperable CRPC). In particular, cancers amenable to treatment with a PD-1 axis binding antagonist (e.g., PD-L1 binding antagonist, e.g., anti-PD-L1 antibody, e.g., atezolizumab) and an antiandrogen (e.g., an AR antagonist, e.g., enzalutamide) include, without limitation, prostate cancer (e.g., castration-resistant prostate cancer (CRPC)), including metastatic CRPC or locally confined, inoperable CRPC. In some instances, the cancer is at an early stage or at a late stage.

The methods described herein are particularly useful in the treatment of a subject with a cancer (e.g., prostate cancer, e.g., CRPC, e.g., mCRPC or locally confined, inoperable CRPC) that is unresponsive to other anti-cancer therapies or in subjects who cannot tolerate, or are ineligible for, other anti-cancer therapies. For example, the subject undergoing treatment for a cancer (e.g., prostate cancer, e.g., CRPC, e.g., mCRPC or locally confined, inoperable CRPC) may have been previously treated with an anti-cancer therapy prior to receiving the treatment method described herein, wherein the subject failed to respond to the previous anti-cancer therapy. In some instances, the subject (e.g., a subject having a cancer, e.g., a prostate cancer, e.g., mCRPC or locally confined, inoperable CRPC) may have received and failed to respond to treatment including an androgen synthesis inhibitor (e.g., abiraterone, orteronel, galeterone, ketoconazole, and/or seviteronel). In some instances, the subject (e.g., a subject having a cancer, e.g., a prostate cancer, e.g., mCRPC or locally confined, inoperable CRPC) failed to respond to treatment including an androgen synthesis inhibitor (abiraterone, orteronel, galeterone, ketoconazole, and/or seviteronel) that had been administered for at least 28 days. Additionally or alternatively, the subject (e.g., a subject having a cancer, e.g., a prostate cancer, e.g., mCRPC or locally confined, inoperable CRPC) may have received and failed to respond to treatment including a taxane regimen (e.g., at least one (e.g., at least two or at least three) dosing cycles of a taxane-containing regimen). In some instances, the subject (e.g., a subject having a cancer, e.g., a prostate cancer, e.g., mCRPC or locally confined, inoperable CRPC) may be ineligible, or refuse, to undergo treatment including a taxane regimen (e.g., at least (e.g., at least two or at least three) dosing cycles of a taxane-containing regimen). In some instances, the prior taxane regimen is for treatment of a hormone-sensitive prostate cancer or a CRPC.

In some instances, the subject has cancer (e.g., prostate cancer, e.g., CRPC, e.g., mCRPC or locally confined, inoperable CRPC) that is resistant to one or more anti-cancer therapies. In some instances, resistance to anti-cancer therapy includes recurrence of cancer or refractory cancer (e.g., prostate cancer, e.g., CRPC, e.g., mCRPC or locally confined, inoperable CRPC). Recurrence may refer to the reappearance of cancer (e.g., a prostate cancer, e.g., CRPC, e.g., mCRPC or locally confined, inoperable CRPC), in the original site or a new site, after treatment. In some instances, resistance to an anti-cancer therapy includes progression of the cancer (e.g., prostate cancer, e.g., CRPC, e.g., mCRPC or locally confined, inoperable CRPC) during or following treatment with the anti-cancer therapy (e.g., during or following a medical or surgical castration). For example, in some instances, the subject may display prostate-specific antigen (PSA) progression (e.g., two or more increases (e.g., 3, 4, or 5 or more increases) in PSA over a previous reference value (e.g., increases in PSA over a previous reference value of 1 ng/ml as the minimum starting value) with each progression measurement at least 1 week apart). In some instances, resistance to a cancer therapy includes cancer (e.g., prostate cancer, e.g., CRPC, e.g., mCRPC or locally confined, inoperable CRPC) that does not respond to treatment (e.g., treatment including an androgen synthesis inhibitor and/or a taxane regimen). The cancer (e.g., prostate cancer, e.g., CRPC, e.g., mCRPC or locally confined, inoperable CRPC) may be resistant at the beginning of treatment (e.g., treatment including an androgen synthesis inhibitor and/or a taxane regimen), or it may become resistant during treatment (e.g., treatment including an androgen synthesis inhibitor and/or a taxane regimen).

### III. Biomarkers

Additionally provided herein are methods for treating a cancer (e.g., prostate cancer, e.g., CRPC, e.g., mCRPC or locally confined, inoperable CRPC) in a subject, wherein treatment is guided by diagnostic methods that involve determining the presence and/or expression levels/amount of one or more biomarkers in a sample obtained from the subject.

Biomarkers can include, but are not limited to, PD-L1 and CD8 expression on tumor tissues, expression of T-effector-associated genes (e.g., CD8A, perforin (PRF1), granzyme A (GZMA), granzyme B (GZMB), interferon-γ (IFN-γ), CXCL9, or CXCL10), activated stroma-associated genes (e.g., transforming growth factor-β (TGF-β), fibroblast-activated protein (FAP), podoplanin (PDPN), a collagen gene, or biglycan (BGN)), myeloid-derived suppressor cell-associated genes (e.g., CD68, CD163, FOXP3, or androgen-regulated gene 1), androgen receptor (AR) gene, germline and somatic mutations from tumor tissue and/or from circulating tumor DNA in blood (including, but not limited to, mutation load, MSI, and MMR defects), identified through WGS and/or NGS, and plasma derived cytokines. In some instances, the PD-L1 biomarker is PD-L1.

In some instances, the method includes determining the presence and/or expression levels/amount of a biomarker (e.g., PD-L1) in a sample from the individual, and administering an effective amount of a PD-1 axis binding antagonist (e.g., PD-L1 binding antagonist, e.g., anti-PD-L1 antibody, e.g., atezolizumab) and/or an antiandrogen (e.g., an AR antagonist, e.g., enzalutamide) to the individual.

In some instances, expression levels or amount of a biomarker (e.g., PD-L1) in a first sample is increased or elevated (e.g., an increase of at least about 1.5-fold, 1.6-fold, 1.8-fold, 2-fold, 3-fold, 4-fold, 5-fold, 6-fold, 7-fold, 8-fold, 9-fold, or 10-fold in expression or amount) as compared to the expression levels or amount in a second sample. In some instances, expression levels or amount of a biomarker (e.g., PD-L1) in a first sample is decreased or reduced (e.g., a decrease of at least about 1.5-fold, 1.6-fold, 1.8-fold, 2-fold, 3-fold, 4-fold, 5-fold, 6-fold, 7-fold, 8-fold, 9-fold, or 10-fold in expression or amount) as compared to expression levels or amount in a second sample. In certain instances, the second sample is a reference sample, reference cell, reference tissue, control sample, control cell, or control tissue. In certain instances, the first sample is a biological sample (e.g., tissue, serum, plasma, whole blood, or urine) obtained from a subject having cancer (e.g., prostate cancer, e.g., CRPC, e.g., mCRPC or locally confined, inoperable CRPC).

In some instances, the presence and/or expression levels/amount of the biomarker (e.g., PD-L1) indicates that the subject is likely to have increased clinical benefit when the subject is treated with the PD-1 axis binding antagonist (e.g., PD-L1 binding antagonist, e.g., anti-PD-L1 antibody, e.g., atezolizumab) and/or an antiandrogen (e.g., an AR antagonist, e.g., enzalutamide). In some instances, the increased clinical benefit comprises a relative increase in one or more of the following: overall survival (OS), progression-free survival (PFS), complete response (CR), partial response (PR) and combinations thereof. Presence and/or expression levels/amount of a biomarker (e.g., PD-L1) can be determined qualitatively and/or quantitatively based on any suitable criterion known in the art, including but not limited to DNA, mRNA, cDNA, proteins, protein fragments and/or gene copy number.

### IV. Pharmaceutical Compositions and Formulations

Pharmaceutical compositions and formulations as described herein can be prepared by mixing more of the active ingredients (e.g., an anti-PD-L1 antibody and/or an antiandrogen (e.g., an AR antagonist, e.g., enzalutamide)) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®; Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The compositions and formulations herein may also contain more than one active ingredients as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films, or microcapsules. The formulations to be used for in vivo administration are generally sterile. Sterility may be readily accomplished, for example, by filtration through sterile filtration membranes.

### V. Articles of Manufacture and Kits

In another aspect of the invention, an article of manufacture or kit containing materials useful for the treatment, prevention, and/or diagnosis of the disorders described above is provided. The article of manufacture or kit may comprise a PD-1 axis binding antagonist (e.g., PD-L1 binding antagonist, e.g., anti-PD-L1 antibody, e.g., atezolizumab) and/or an antiandrogen (e.g., an AR antagonist, e.g., enzalutamide). In some instances, the article of manufacture or kit further comprises a package insert comprising instructions for using the PD-1 axis binding antagonist (e.g., PD-L1 binding antagonist, e.g., anti-PD-L1 antibody, e.g., atezolizumab) in conjunction with an antiandrogen (e.g., an AR antagonist, e.g., enzalutamide) to treat or delay progression of cancer (e.g., prostate cancer, e.g., CRPC, e.g., mCRPC or locally confined, inoperable CRPC) in a subject. Any of the PD-1 axis binding antagonists (e.g., PD-L1 binding antagonist, e.g., anti-PD-L1 antibody, e.g., atezolizumab) and/or antiandrogens (e.g., AR antagonist, e.g., enzalutamide) described herein may be included in the article of manufacture or kits.

In some instances, the article of manufacture or kit includes a PD-1 axis binding antagonist (e.g., PD-L1 binding antagonist, e.g., anti-PD-L1 antibody, e.g., atezolizumab) and a package insert including instructions for administration of the PD-1 axis binding antagonist (e.g., PD-L1 binding antagonist, e.g., anti-PD-L1 antibody, e.g., atezolizumab) in combination with an antiandrogen (e.g., AR antagonist, e.g., enzalutamide) for treating a subject having cancer (e.g., prostate cancer, e.g., CRPC, e.g., mCRPC or locally confined, inoperable CRPC).

In some instances, the article of manufacture or kit includes a first medicament including a PD-1 axis binding antagonist (e.g., PD-L1 binding antagonist, e.g., anti-PD-L1 antibody, e.g., atezolizumab), a second medicament including an antiandrogen (e.g., AR antagonist, e.g., enzalutamide), and a package insert including instructions for administration of the first medicament and the second medicament for treating a subject having cancer (e.g., prostate cancer, e.g., CRPC, e.g., mCRPC or locally confined, inoperable CRPC).

In some instances, the article of manufacture or kit includes an antiandrogen (e.g., AR antagonist, e.g., enzalutamide) and a package insert including instructions for administration of the antiandrogen (e.g., AR antagonist, e.g., enzalutamide) in combination with a PD-1 axis binding antagonist (e.g., PD-L1 binding antagonist, e.g., anti-PD-L1 antibody, e.g., atezolizumab) for treating a subject having cancer (e.g., prostate cancer, e.g., CRPC, e.g., mCRPC or locally confined, inoperable CRPC).

In some embodiments, the article of manufacture comprises a container and a label or package insert on, or associated with, the container. In some embodiments, the anti-PD-L1 antibody (e.g., atezolizumab) and the antiandrogen (e.g., an AR antagonist, e.g., enzalutamide) are in the same container or separate containers. Suitable containers include, for example, bottles, vials, syringes, IV solution bags, etc. The containers may be formed from a variety of materials such as glass or plastic. In some embodiments, the container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an anti-PD-L1 antibody described herein. The label or package insert indicates that the composition is used for treating the condition of choice (e.g., a cancer, e.g., prostate cancer, e.g., CRPC, e.g., mCRPC or locally confined CROP) and further includes information related to at least one of the dosing regimens described herein. Moreover, the article of manufacture may comprise (a) a first container with a composition contained therein, wherein the composition comprises an anti-PD-L1 antibody described herein (e.g., atezolizumab); and (b) a second container with a composition contained therein, wherein the composition comprises an antiandrogen (e.g., an AR antagonist, e.g., enzalutamide), and, optionally, a cytotoxic agent or an otherwise therapeutic agent. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### VI. Example

The following is an example of the methods of the invention. It is understood that various other embodiments may be practiced, given the general description provided above.

### Example 1. Efficacy of Anti-PD-L1 Antibody in Combination with Enzalutamide in Patients with Castration-Resistant Prostate Cancer (CRPC)

To evaluate the safety and efficacy of treatment with an anti-PD-L1 antibody (atezolizumab (MPDL3280A)) in combination with an antiandrogen (enzalutamide) compared with enzalutamide alone in patients with prostate cancer (e.g., CRPC (e.g., metastatic or locally confined, inoperable CRPC)), patients are enrolled in a phase III, multicenter, randomized, open-label study. To be eligible, patients must (i) have previously failed treatment with an androgen synthesis inhibitor (e.g., progressed during treatment with an androgen synthesis inhibitor (e.g., abiraterone)) for prostate cancer (e.g., CRPC (e.g., metastatic or locally confined, inoperable CRPC)) and (ii) have failed treatment with a taxane regimen (e.g., a taxane regimen for prostate cancer (e.g., metastatic hormone-sensitive and/or CRPC (e.g., mrCPRC))), were ineligible for treatment with a taxane regimen for prostate cancer (e.g., CRPC, e.g., mCRPC), or refused treatment with a taxane regimen for prostate cancer (e.g., CRPC, e.g., mCRPC). The clinical trial consists of two phases, a safety run-in phase and a randomized phase, as described in detail below.

Safety Run-In Phase (Phase 1)

First, the trial includes a safety run-in phase. A safety run-in phase is incorporated into the study design to evaluate the preliminary safety profile of atezolizumab in combination with enzalutamide prior to initiating the randomized phase of the study. Currently, no safety data is available for the combination of atezolizumab and enzalutamide. Based on the different mechanism of action for each product, the overlapping risks of enzalutamide and atezolizumab are thought to be minimal and are not expected to significantly increase the incidence of adverse events seen in monotherapy studies.

In the safety run-in phase of the study, 10 patients receive atezolizumab in combination with enzalutamide. After 10 patients are enrolled, enrollment is stopped temporarily and the 10 patients are monitored closely for adverse events until the last patient has completed the first cycle (21 days). To monitor patients for adverse events, the patients are evaluated for clinically relevant toxicities that are known to be associated with atezolizumab and/or enzalutamide. These adverse events include, but are not limited to, gastrointestinal toxicities (e.g., diarrhea and immune-related colitis), immune-related hepatitis, immune-related pancreatitis, immune-related pneumonitis, immune-related endocrinopathies (e.g., diabetes mellitus, hypothyroidism, hyperthyroidism, or adrenal insufficiency), and neurologic disorders (e.g., immune-related meningoencephalitis, immune-related neuropathies (e.g., myasthenic syndrome and/or myasthenia gravis, Guillain-Barré syndrome), vertigo and/or dizziness, falls, seizures, or posterior reversible encephalopathy syndrome (PRES)).

After 10 patients receive study treatment and complete at least one dosing cycle (21 days), the data are evaluated to assess the safety of the combination treatment prior to initiation of the randomized phase of the study. To evaluate the data from the safety run-in phase, the type and frequency of adverse events observed are compared to the type and frequency of events previously described in studies with atezolizumab or enzalutamide. On the basis of this evaluation, the study can enroll an additional 10 patients into the safety run-in portion of the study. Furthermore, in some instances, the observation period is extended until the last patient has completed two cycles (42 days), or the frequency of periodic safety monitoring during the randomized phase of the study can increase.

Randomization Phase (Phase 2)

In this second phase of the study, patients are randomized to one of two treatment arms in a 1:1 ratio (experimental arm to control arm). In the experimental arm, patients receive atezolizumab in combination with enzalutamide. In the control arm, patients receive enzalutamide alone. The randomization is stratified on the basis of prior taxane-containing regimen (e.g., patient received at least one cycle of a taxane-containing regimen) for prostate cancer (e.g., CRPC, e.g., mCRPC), pain severity (e.g., Brief Pain Inventory Question assessing pain at its worst over the past 24 hours, presence of liver metastasis, and serum lactate dehydrogenase (LDH) level (e.g., LDH upper limit of normal (ULN) vs. >ULN). These stratification factors have been identified as critical prognostic factors for patients with mCRPC. In particular, the magnitude of benefit in patients treated with a hormonally-based therapy may be attenuated after a taxane-containing therapy.

During treatment, patients receive a fixed dose of 1200 mg atezolizumab (equivalent to an average body weight-based dose of 15 mg/kg) administered by intravenous infusion every 3 weeks (21±3 days). Atezolizumab is administered on Day 1 of each dosing cycle. Enzalutamide is administered orally at a dose of 160 mg (four 40 mg capsules) once daily. Treatment is continued until lack of clinical benefit, worsening of symptoms, decline in performance status, or tumor progression at a critical site that cannot be managed with protocol-accepted therapy.

Biomarkers

Patient samples, including archival tumor tissues, as well as urine, serum, plasma and whole blood, are collected for exploratory biomarker assessments for all patients in the randomized phase. In addition assessing PD-L1 status, biomarkers related to resistance, disease progression, and clinical benefit of atezolizumab and/or enzalutamide are analyzed. For example, potential predictive and prognostic biomarkers related to the clinical benefit and safety of atezolizumab and/or enzalutamide are analyzed.

Tumor tissue and blood samples collected at baseline (and, if deemed clinically feasible by the investigator, tumor tissue collected at the time of disease progression) enables whole-exome sequencing (WES) and/or next-generation sequencing (NGS) to identify somatic mutations that are predictive of response to study treatment, are associated with progression to a more severe disease state, are associated with acquired resistance to study treatment, are associated with susceptibility to developing adverse events, or can increase the knowledge and understanding of disease biology.

Biomarkers include, but are not limited to, PD-L1 and CD8 expression on tumor tissues, expression of T-effector-associated genes (e.g., CD8A, perforin (PRF1), granzyme A (GZMA), granzyme B (GZMB), interferon-γ (IFN-γ), CXCL9, or CXCL10), activated stroma-associated genes (e.g., transforming growth factor-β (TGF-β), fibroblast-activated protein (FAP), podoplanin (PDPN), a collagen gene, or biglycan (BGN)), myeloid-derived suppressor cell-associated genes (e.g., CD68, CD163, FOXP3, or androgen-regulated gene 1), androgen receptor (AR) gene, germline and somatic mutations from tumor tissue and/or from circulating tumor DNA in blood (including, but not limited to, mutation load, MSI, and MMR defects), identified through WGS and/or NGS, and plasma derived cytokines.

Concomitant Therapy

Certain concomitant therapies are permitted. Concomitant therapies include any medication (e.g., prescription drugs, over the counter drugs, vaccines, herbal or homeopathic remedies, nutritional supplements) used by a patient in addition to protocol-mandated study treatment from seven days prior to initiation of study treatment to the treatment discontinuation visit.

Patients are permitted to use the following concomitant therapies during the study. Patients who have not undergone bilateral orchiectomy must be maintained on a GnRH analog or GnRH antagonist throughout the study (i.e., both treatment phase and follow up). Further, patients are permitted to use prophylactic or therapeutic anticoagulation therapy (e.g., low molecular weight heparin; for potential drug-drug interaction of enzalutamide and warfarin), inactivated influenza vaccinations, mineralocorticoids (e.g., fludrocortisone), corticosteroids administered for COPD or asthma, low dose corticosteroids administered for orthostatic hypotension or adrenocortical insufficiency, standard of care corticosteroid use of no greater than the equivalent of 10 mg of prednisone or prednisolone per day, palliative surgical procedures to treat skeletal related events, focal palliative radiotherapy (e.g., external beam radiotherapy to address single sites of disease). In addition, atezolizumab and enzalutamide treatment can continue during palliative radiotherapy.

Patients receiving bisphosphonates or denosumab prior to enrollment are maintained on bisphosphonate or denosumab therapy during screening and while actively treated with study drug. Initiation of bisphosphonates or denosumab is discouraged during the treatment phase of the study due to potential immunomodulatory properties. However, initiation of such treatment should not result in discontinuation of study treatment.

Blood transfusions are allowed throughout the study. In some instances, premedication with antihistamines, antipyretics, and/or analgesics are administered for the second and subsequent atezolizumab infusions only, at the discretion of the investigator. In general, investigators can manage a patient's care with supportive therapies as clinically indicated, per local standard practice. Patients who experience infusion associated symptoms can receive treatment symptomatically with acetaminophen, ibuprofen, diphenhydramine, and/or H2 receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and 132 adrenergic agonists).

Efficacy Endpoints

To evaluate the efficacy of atezolizumab and enzalutamide compared with enzalutamide alone, overall survival (e.g., the time from randomization to death from any cause) is measured as an endpoint. Efficacy endpoints can further include overall survival probability at 12 or 24 months, time to cancer-related pain progression while receiving study treatment, time to first symptomatic skeletal event (SSE), radiographic progression-free survival (rPFS, e.g., time from randomization to the earliest occurrence of progression by bone scan, progression of soft tissue lesions, or death from any cause), rPFS probability at 6 or 12 months, and immune-modified rPFS (e.g., time from randomization to the earliest occurrence of disease progression detected by bone scan, progression of soft tissue lesions, or death from any cause). Other efficacy endpoints can include time to initiation or increased opiate analgesic use for cancer pain while receiving study treatment, prostate-specific antigen (PSA) response rate (e.g., >50% decrease in PSA from baseline that is confirmed after 3 weeks by a consecutive confirmatory PSA measurement), time to PSA progression (e.g., time from randomization to the time of PSA progression), objective response rate in soft tissue lesions (e.g., the proportion of patients with either a CR or PR on two consecutive occasions 6 weeks apart). Yet further efficacy endpoints can include duration of response in soft tissue lesions, disease control rate, modified progression-free survival, time to unequivocal clinical progression, time to initiation of next systemic anti-cancer therapy, physical function, health-related quality of life, symptoms associated with prostate cancer, time to pain palliation, health status as measured using the EuroQol 5-Dimension, 5-Level Questionnaire (EQ-5D-5L) questionnaire for health economic modeling, and/or tolerability of enzalutamide, with or without atezolizumab.

Pharmacokinetic Analyses

To characterize the pharmacokinetics of atezolizumab when given in combination with enzalutamide, serum concentrations of atezolizumab are determined from subjects at different time points. Further, to characterize the pharmacokinetics of enzalutamide and its active metabolite N-desmethyl enzalumatide when enzalutamide is administered alone or in combination with atezolizumab, plasma concentration of enzalutamide and/or N-desmethyl enzalutamide is obtained from subjects at different time points in the safety run-in phase in a PK cohort in the randomized phase. PK analyses are reported and summarized using descriptive statistics.

### VII. Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method for treating a subject having a prostate cancer comprising administering to the subject an effective amount of an anti-PD-L1 antibody and an antiandrogen in one or more dosing cycles.

2. The method of claim 1, wherein the antiandrogen is an androgen receptor (AR) antagonist.

3. The method of claim 2, wherein the AR antagonist is a non-steroidal AR antagonist.

4. The method of claim 3, wherein the non-steroidal AR antagonist is enzalutamide.

5. The method of claim 4, wherein the method comprises administering enzalutamide at a dose of between about 80 mg to about 240 mg.

6. The method of claim 5, wherein the method comprises administering enzalutamide at a dose of about 160 mg.

7. The method of claim 6, wherein the method comprises administering enzalutamide at a dose of about 160 mg on each day of the one or more dosing cycles.

8. The method of any one of claims 1-7, wherein the anti-PD-L1 antibody inhibits the binding of PD-L1 to PD-1, the binding of PD-L1 to B7-1, or the binding of PD-L1 to both PD-1 and B7-1.

9. The method of any one of claims 1-8, wherein the anti-PD-L1 antibody is selected from the group consisting of atezolizumab (MPDL3280A), YW243.55.570, MSB0010718C, MDX-1105, and MED14736.

10. The method of any one of claims 1-9, wherein the anti-PD-L1 antibody comprises the following hypervariable regions (HVRs):
(a) the HVR-H1 sequence is
(SEQ ID NO: 1)
GFTFSDSWIH;
(b) the HVR-H2 sequence is
(SEQ ID NO: 2)
AWISPYGGSTYYADSVKG;
(c) the HVR-H3 sequence is
(SEQ ID NO: 3)
RHWPGGFDY;
(d) the HVR-L1 sequence is
(SEQ ID NO: 4)
RASQDVSTAVA;
(d) the HVR-L2 sequence is
(SEQ ID NO: 5)
SASFLYS;
and
(f) the HVR-L3 sequence is
(SEQ ID NO: 6)
QQYLYHPAT.

11. The method of claim 10, wherein the anti-PD-L1 antibody comprises:
(a) a heavy chain variable (VH) domain comprising an amino acid sequence having at least 95% sequence identity to the amino acid sequence of SEQ ID NO: 7;
(b) a light chain variable (VL) domain comprising an amino acid sequence having at least 95% sequence identity to the amino acid sequence of SEQ ID NO: 8; or
(c) a VH domain as in (a) and a VL domain as in (b).

12. The method of claim 11, wherein the anti-PD-L1 antibody comprises:
(a) a VH domain comprising the amino acid sequence of SEQ ID NO: 7;
(b) a VL domain comprising the amino acid sequence of SEQ ID NO: 8; or
(c) a VH domain as in (a) and a VL domain as in (b).

13. The method of claim 12, wherein the anti-PD-L1 antibody comprises:
(a) a VH domain comprising the amino acid sequence of SEQ ID NO: 7; and
(b) a VL domain comprising the amino acid sequence of SEQ ID NO: 8.

14. The method of claim 13, wherein the anti-PD-L1 antibody is atezolizumab.

15. The method of claim 14, wherein the method comprises administering atezolizumab at a dose of between about 600 mg to about 1800 mg.

16. The method of claim 15, wherein the method comprises administering atezolizumab at a dose of about 1200 mg.

17. The method of claim 14, wherein the method comprises administering atezolizumab at a dose of about 5 mg/kg to about 20 mg/kg.

18. The method of claim 17, wherein the method comprises administering atezolizumab at a dose of about 15 mg/kg.

19. The method of any one of claims 14-18, wherein the method comprises administering atezolizumab at a fixed dose.

20. The method of any one of claims 1-19, wherein the method comprises administering the anti-PD-L1 antibody on about Day 1 of each of the one or more dosing cycles.

21. The method of any one of claims 1-20, wherein the length of each of the one or more dosing cycles is 18-24 days.

22. The method of claim 21, wherein the length of each of the one or more dosing cycles is 21 days.

23. The method of any one of claims 1-22, wherein the method comprises administering the antiandrogen before the anti-PD-L1 antibody, simultaneous with the anti-PD-L1 antibody, or after the anti-PD-L1 antibody.

24. The method of any one of claims 1-23, wherein the method comprises administering the anti-PD-L1 antibody intravenously, intramuscularly, subcutaneously, topically, orally, transdermally, intraperitoneally, intraorbitally, by implantation, by inhalation, intrathecally, intraventricularly, or intranasally.

25. The method of claim 24, wherein the method comprises administering the anti-PD-L1 antibody intravenously.

26. The method of any one of claims 1-25, wherein the method comprises administering the antiandrogen orally, intravenously, intramuscularly, subcutaneously, topically, transdermally, intraperitoneally, intraorbitally, by implantation, by inhalation, intrathecally, intraventricularly, or intranasally.

27. The method of claim 26, wherein the method comprises administering the antiandrogen orally.

28. The method of any one of claims 1-27, further comprising determining the expression level of a biomarker in a sample from the subject.

29. The method of claim 28, wherein the biomarker is a T-effector-associated gene, an activated stroma-associated gene, or a myeloid-derived suppressor cell-associated gene.

30. The method of claim 29, wherein the T-effector-associated gene is CD8A, perforin (PRF1), granzyme A (GZMA), granzyme B (GZMB), interferon-γ (IFNγ), CXCL9, or CXCL10.

31. The method of claim 29, wherein the activated stroma-associated gene is transforming growth factor-β (TGF-β), fibroblast-activated protein (FAP), podplanin (PDPN), a collagen gene, or biglycan (BGN).

32. The method of claim 29, wherein the myeloid-derived suppressor cell-associated gene is CD68, CD163, FOXP3, or androgen-regulated gene 1.

33. The method of claim 28, wherein the biomarker is PD-L1, CD8, or androgen receptor (AR) gene.

34. The method of claim 33, wherein the biomarker is PD-L1.

35. The method of any one of claims 28-34, wherein a change in the expression level of the biomarker relative to a reference level is predictive of the subject's likelihood to respond to the treatment.

36. The method of any one of claims 1-35, wherein the prostate cancer is a castration-resistant prostate cancer (CRPC).

37. The method of claim 36, wherein the CRPC is a metastatic CRPC.

38. The method of claim 36, wherein the CRPC is a locally confined and inoperable CRPC.

39. The method of any one of claims 1-38, wherein the subject failed to respond to a previous treatment comprising an androgen synthesis inhibitor.

40. The method of claim 39, wherein the subject failed to respond to a previous treatment comprising an androgen synthesis inhibitor and a taxane regimen.

41. The method of claim 39, wherein the subject failed to respond to a previous treatment comprising an androgen synthesis inhibitor and is ineligible for, or refuses treatment with, a taxane regimen.

42. The method of claim 40 or 41, wherein the taxane regimen is for treatment of a hormone-sensitive prostate cancer or a CRPC.

43. The method of any one of claims 39-42, wherein the previous treatment comprising the androgen synthesis inhibitor was at least 28 days.

44. The method of any one of claims 39-43, wherein the androgen synthesis inhibitor is abiraterone, orteronel, galeterone, ketoconazole, or seviteronel.

45. A kit comprising an anti-PD-L1 antibody and a package insert comprising instructions for administration of the anti-PD-L1 antibody in combination with an antiandrogen for treating a subject having a prostate cancer.

46. A kit comprising a first medicament comprising an anti-PD-L1 antibody, a second medicament comprising an antiandrogen, and a package insert comprising instructions for administration of the first medicament and the second medicament for treating a subject having a prostate cancer.

47. A kit comprising an antiandrogen and a package insert comprising instructions for administration of the antiandrogen in combination with an anti-PD-L1 antibody for treating a subject having a prostate cancer.

48. The kit of any one of claims 45-47, wherein the antiandrogen is an androgen receptor (AR) antagonist.

49. The kit of claim 48, wherein the AR antagonist is a non-steroidal AR antagonist.

50. The kit of claim 49, wherein the non-steroidal AR antagonist is enzalutamide.

51. The kit of claim 50, wherein enzalutamide is formulated for administration at a dose of between about 80 mg to about 240 mg.

52. The kit of claim 51, wherein enzalutamide is formulated for administration at a dose of about 160 mg.

53. The kit of claim 52, wherein enzalutamide is formulated for administration at a dose of about 160 mg on each day of the one or more dosing cycles.

54. The kit of any one of claims 45-53, wherein the anti-PD-L1 antibody inhibits the binding of PD-L1 to PD-1, the binding of PD-L1 to B7-1, or the binding of PD-L1 to both PD-1 and B7-1.

55. The kit of any one of claims 45-54, wherein the anti-PD-L1 antibody is selected from the group consisting of atezolizumab (MPDL3280A), YW243.55.S70, MSB0010718C, MDX-1105, and MED14736.

56. The kit of any one of claims 45-55, wherein the anti-PD-L1 antibody comprises the following hypervariable regions (HVRs):
(a) the HVR-H1 sequence is
(SEQ ID NO: 1)
GFTFSDSWIH;
(b) the HVR-H2 sequence is
(SEQ ID NO: 2)
AWISPYGGSTYYADSVKG;
(c) the HVR-H3 sequence is
(SEQ ID NO: 3)
RHWPGGFDY;
(d) the HVR-L1 sequence is
(SEQ ID NO: 4)
RASQDVSTAVA;
(d) the HVR-L2 sequence is
(SEQ ID NO: 5)
SASFLYS;
and
(f) the HVR-L3 sequence is
(SEQ ID NO: 6)
QQYLYHPAT.

57. The kit of claim 56, wherein the anti-PD-L1 antibody comprises:
(a) a heavy chain variable (VH) domain comprising an amino acid sequence having at least 95% sequence identity to the amino acid sequence of SEQ ID NO: 7;
(b) a light chain variable (VL) domain comprising an amino acid sequence having at least 95% sequence identity to the amino acid sequence of SEQ ID NO: 8; or
(c) a VH domain as in (a) and a VL domain as in (b).

58. The kit of claim 57, wherein the anti-PD-L1 antibody comprises:
(a) a VH domain comprising the amino acid sequence of SEQ ID NO: 7;
(b) a VL domain comprising the amino acid sequence of SEQ ID NO: 8; or
(c) a VH domain as in (a) and a VL domain as in (b).

59. The kit of claim 58, wherein the anti-PD-L1 antibody comprises:
(a) a VH domain comprising the amino acid sequence of SEQ ID NO: 7; and
(b) a VL domain comprising the amino acid sequence of SEQ ID NO: 8.

60. The method of claim 59, wherein the anti-PD-L1 antibody is atezolizumab.

61. The kit of claim 60, wherein atezolizumab is formulated for administration at a dose of between about 600 mg to about 1800 mg.

62. The kit of claim 61, wherein atezolizumab is formulated for administration at a dose of about 1200 mg.

63. The kit of claim 62, wherein atezolizumab is formulated for administration at a dose of about 5 mg/kg to about 20 mg/kg.

64. The kit of claim 63, wherein atezolizumab is formulated for administration at a dose of about 15 mg/kg.

65. The kit of any one of claims 60-64, wherein atezolizumab is formulated for administration at a fixed dose.

66. The kit of any one of claims 45-65, wherein the prostate cancer is a CRPC.

67. The kit of claim 66, wherein the CRPC is a metastatic CRPC.

68. The kit of claim 66, wherein the CRPC is a locally confined and inoperable CRPC.

69. The kit of any one of claims 45-68, wherein the subject failed to respond to a previous treatment comprising an androgen synthesis inhibitor.

70. The kit of claim 69, wherein the subject failed to respond to a previous treatment comprising an androgen synthesis inhibitor and a taxane regimen.

71. The kit of claim 69, wherein the subject failed to respond to a previous treatment comprising an androgen synthesis inhibitor and is ineligible for, or refuses treatment with, a taxane regimen.

72. The kit of claim 70 or 71, wherein the taxane regimen is for treatment of a hormone-sensitive prostate cancer or a CRPC.

73. The kit of any one of claims 69-72, wherein the previous treatment comprising the androgen synthesis inhibitor was at least 28 days.

74. The kit of any one of claims 69-73, wherein the androgen synthesis inhibitor is abiraterone, orteronel, galeterone, ketoconazole, or seviteronel.
